(19) United States
(12) Patent Application Publication
(10) Pub. No.: US 2016/0242687 A1
(43) Pub. Date: Aug. 25, 2016

(54) DETECTION METHOD USING DETECTION ELEMENT, DETECTION ELEMENT, MEASUREMENT DEVICE, AND INSULIN SUPPLY DEVICE

(71) Applicant: Seiko Epson Corporation, Tokyo (JP)

(72) Inventors: Tetsuji FUJITA, Chino (JP); Yasuhiro MOMOSE, Matsumoto (JP)

(21) Appl. No.: 15/019,143

(22) Filed: Feb. 9, 2016

(30) Foreign Application Priority Data
Feb. 20, 2015 (JP) 2015-031435

(51) Int. Cl. A61B 5/1486; A61B 5/00; A61M 5/172; A61B 5/145

(52) U.S. Cl. CPC A61B 5/14865; A61B 5/1451; A61B 5/14532; A61B 5/742; A61M 5/1723; A61M 2230/201; A61M 2205/50

(57) ABSTRACT

A detection method using a detection element, including a first electrode and a second electrode, in which a detection layer containing an enzyme is provided in the first electrode and the second electrode, includes a first step of measuring an electric current generated by decomposition of hydrogen peroxide generated from glucose in the detection layer of the first electrode as an electric current value between the first electrode and the second electrode, and a second step of measuring an electric current generated by decomposition of hydrogen peroxide generated from glucose in the detection layer of the second electrode as an electric current value between the first electrode and the second electrode.

### BACKGROUND

1. Technical Field

The present invention relates to a detection method using a detection element, a detection element, a measurement device, and an insulin supply device.

2. Related Art

Diabetic patients are divided into type I and type II according to the symptoms, however, in both types, insulin secretion from the pancreas is not normal, and therefore, the internal organs cannot normally incorporate glucose to cause metabolic abnormality and also to decrease the body weight. Moreover, it is known that when a state where the blood glucose level is high is maintained over a long period of time, serious complications such as “diabetic retinopathy”, “diabetic nephropathy”, “diabetic microvascular disease”, and “diabetic neuropathy” are developed. The current situation is that in order to prevent the development of such serious complications, a treatment method in which the blood glucose level is maintained within a normal range by administering insulin by injection is adopted for patients in whom a state where the blood glucose level is high persists.

Here, in type I diabetic patients, insulin is not secreted at all due to a pancreatic disease, and therefore, it is necessary to measure the blood glucose level by blood collection several times a day (at least four times, before meal and before going to bed), and administer insulin. As for the timing of administration, in order to suppress an excess increase in the blood glucose level after meal, the blood glucose level before meal is measured and the calories of the meal is calculated beforehand, and then the dose of insulin is determined and insulin is administered by injection before meal. Further, what is known as an increase in the blood glucose level is a symptom called “dawn phenomenon”, and the blood glucose level increases at dawn at 8 to 10 hours after going to bed. However, in order to deal with this physiological phenomenon, it is necessary to wake up once before dawn and measure the blood glucose level by blood collection, and if the blood glucose level is high, it is necessary to administer insulin. In this manner, general healthy individuals can live a daily life without caring about the blood glucose level or the like, however, diabetic patients (particularly type I diabetic patients) are compelled to live a life while caring about the blood glucose level all day long.

The current situation is that in order to reduce the burden of daily life on such patients and families who support the patients, that is, in order to improve the QOL of the patients and families of the patients, the development of an artificial pancreas or a device equivalent thereto has been demanded. In view of this, first, it is necessary to continuously and automatically measure and control the blood glucose level.

For example, a device so-called a continuous glucose monitor (CGM) device for use in monitoring the glucose level in the interstitial fluid of a patient over a long period of time by embedding a blood glucose level sensor in the body (subcutaneous tissue) has been proposed (see, for example, JP-T-2006-502810 (Patent Document 1) and JP-T-2014-504521 (Patent Document 2)). In a technique for the quantitative determination of glucose used in these Patent Documents 1 and 2, an enzymatic reaction is utilized.

The basic principle of the quantitative determination of glucose using an enzymatic reaction is that in the presence of an enzyme (for example, glucose oxidase), when glucose and oxygen are present in the vicinity of the enzyme, gluconic acid and hydrogen peroxide are generated. By measuring the amount of an electric current generated by electrically decomposing the generated hydrogen peroxide, the amount of hydrogen peroxide can be quantitatively determined, and therefore, the glucose level can be calculated based on this.

By using such an enzymatic reaction, it is possible to monitor the blood glucose level continuously with the sensor embedded in the body. However, the current situation is that with the use of the currently available continuous glucose monitor (CGM), the behavior of whether the blood glucose level increases or decreases over time with respect to the blood glucose level when the measurement is started can be determined, but it cannot be said that the magnitude of a blood glucose level can be measured over time with high accuracy because of a too large drift phenomenon. Due to this, the currently available continuous glucose monitor (CGM) cannot be used as a device for determining when to administer insulin.

### SUMMARY

An advantage of some aspects of the invention is to provide a detection method using a detection element capable of measuring the magnitude of a blood glucose level over time with high accuracy by suppressing the occurrence of a drift phenomenon in a continuous glucose monitor (CGM), a detection element capable of achieving the detection method using a detection element, and also a measurement device and an insulin supply device, each including this detection element.

The advantage can be achieved by the aspects of the invention described below.

A detection method according to an aspect of the invention is a detection method using a detection element including a first electrode and a second electrode, in which a detection layer containing an enzyme is provided in the first electrode and the second electrode, the method including: a first step of measuring an electric current generated by decomposition of hydrogen peroxide generated from glucose in the detection layer of the first electrode as an electric current value between the first electrode and the second electrode, and a second step of measuring an electric current generated by decomposition of hydrogen peroxide generated from glucose in the detection layer of the second electrode as an electric current value between the first electrode and the second electrode.

According to this configuration, in a glucose detection element, a glucose level can be measured with high accuracy by suppressing a drift phenomenon.

In the detection method using a detection element according to the aspect of the invention, it is preferred that the glucose is one permeating into the detection layer from the interstitial fluid in the subcutaneous tissue and the skin.

According to this configuration, the detection of glucose in the interstitial fluid can be continuously performed over a long period of time.

In the detection method using a detection element according to the aspect of the invention, it is preferred that the level of glucose contained in the interstitial fluid is detected based on the electric current value.

According to this configuration, the level of glucose contained in the interstitial fluid can be calculated with high accuracy.

In the detection method using a detection element according to the aspect of the invention, it is preferred that the enzyme is glucose oxidase which decomposes the glucose, oxygen, and water to gluconic acid and the hydrogen peroxide.

By using glucose oxidase, hydrogen peroxide can be generated from glucose in the presence of O₂ and H₂O.

In the detection method using a detection element according to the aspect of the invention, it is preferred that the detection layer is provided in each of the first electrode and the second electrode.

According to this configuration, each of the first electrode and the second electrode has a function of a working electrode and a function of a counter electrode.

In the detection method using a detection element according to the aspect of the invention, it is preferred that the areas of the detection layers of the first electrode and the second electrode have substantially the same size.

According to this configuration, each of the first electrode and the second electrode has a function of a working electrode and a function of a counter electrode.

In the detection method using a detection element according to the aspect of the invention, it is preferred that a third electrode which decomposes hydrogen peroxide by applying a substantially constant voltage between the third electrode and the first electrode is included.

According to this configuration, the decomposition of hydrogen peroxide can be accelerated.

In the detection method using a detection element according to the aspect of the invention, it is preferred that switching between the first step and the second step is performed by a reversing switch which electrically reverses the first electrode and the second electrode.

According to this configuration, switching between the first state and the second state can be reliably performed.

In the detection method using a detection element according to the aspect of the invention, it is preferred that the electric current value is measured a plurality of times at intervals of a fixed time, and switching between the first step and the second step is performed every time when the electric current value is measured.

According to this configuration, a glucose level in the interstitial fluid can be measured with high accuracy by suppressing the occurrence of a drift phenomenon.

In the detection method using a detection element according to the aspect of the invention, it is preferred that the electric current value is measured a plurality of times at intervals of a fixed time, and the plurality of times is determined to be a fixed number of times.

According to this configuration, a glucose level in the interstitial fluid can be measured with high accuracy by suppressing the occurrence of a drift phenomenon.

A detection element according to an aspect of the invention includes a substrate; a first electrode and a second electrode provided on the substrate; a detection layer containing an enzyme and provided in the first electrode and the second electrode; and a reversing switch which electrically reverses the first electrode and the second electrode, wherein an electric current generated by decomposition of hydrogen peroxide generated from glucose in the detection layer is measured as an electric current value between the first electrode and the second electrode.

According to this configuration, in the glucose detection element, a glucose level can be measured with high accuracy by suppressing a drift phenomenon.

A measurement device according to an aspect of the invention preferably includes the detection element according to the aspect of the invention, a calculation section which calculates a glucose level from an electric current value measured by the detection element, and a display section which displays the glucose level.

This measurement device has high reliability.

An insulin supply device according to an aspect of the invention includes the detection element according to the aspect of the invention, a calculation section which calculates a glucose level from an electric current value measured by the detection element, and a supply section which supplies insulin to the subcutaneous tissue and into the skin based on the glucose level.

This insulin supply device has high reliability.

### BRIEF DESCRIPTION OF THE DRAWINGS

The invention will be described with reference to the accompanying drawings, wherein like numbers reference like elements.

FIGS. 1A and 1B are perspective views each schematically showing a state where a detection element according to the invention is attached to a measurement device.

FIG. 2 is a side view showing a state where the detection element according to the invention is attached to the skin.

FIG. 3 is an enlarged vertical cross-sectional view showing a cannula included in the detection element according to the invention.

FIG. 4 is a plan view showing a detection section included in the detection element according to the invention.

FIG. 5 is a vertical cross-sectional view showing the detection section included in the detection element according to the invention.

FIG. 6 is a vertical cross-sectional view showing a configuration example of the detection element according to the invention.

FIG. 7 is a vertical cross-sectional view showing a configuration example of the detection element according to the invention.

FIG. 8 is a view schematically showing a configuration of a circuit according to the invention.

FIG. 9 is a view showing the behavior of the pH value and the amount of O₂ after measuring a glucose level in a counter electrode and a working electrode.

FIG. 10 is a graph showing a relationship between the amount of hydrogen peroxide and the time when a glucose level is repeatedly measured at intervals of a fixed time T by a detection section included in the detection element according to the invention.

FIG. 11 is a flow chart showing a method for repeatedly measuring a glucose level at intervals of a fixed time T by the detection element according to the invention.

FIG. 12 is a perspective view schematically showing a state where the detection element according to the invention is attached to an insulin supply device.

FIG. 13 is a vertical cross-sectional view showing the detection element produced in Example.

FIG. 14 is a graph showing a relationship between the time and the electric current value measured by the detection elements of Example 1 and Comparative Example 1.

### DESCRIPTION OF EXEMPLARY EMBODIMENTS

Hereinafter, a detection method using a detection element, a detection element, a measurement device, and an insulin supply device according to the invention will be described in detail based on embodiments shown in the accompanying drawings.

### Measurement Device

First, prior to the description of a detection method using a detection element according to the invention, a measurement device including a detection element according to the invention will be described.

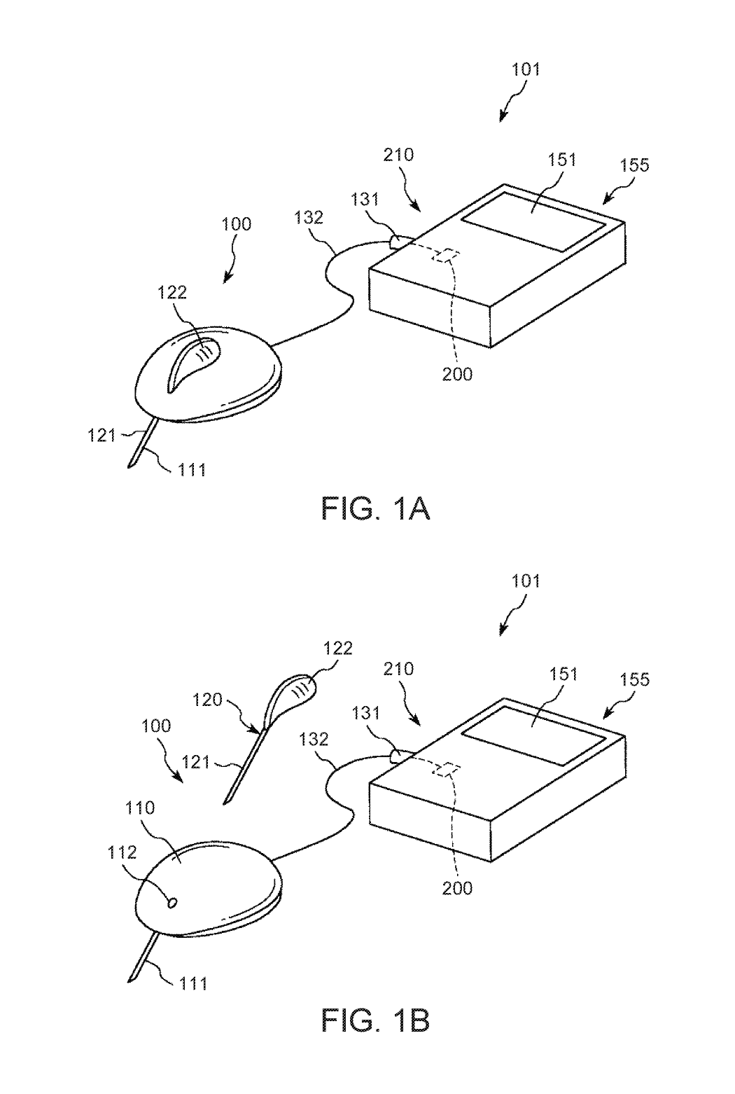

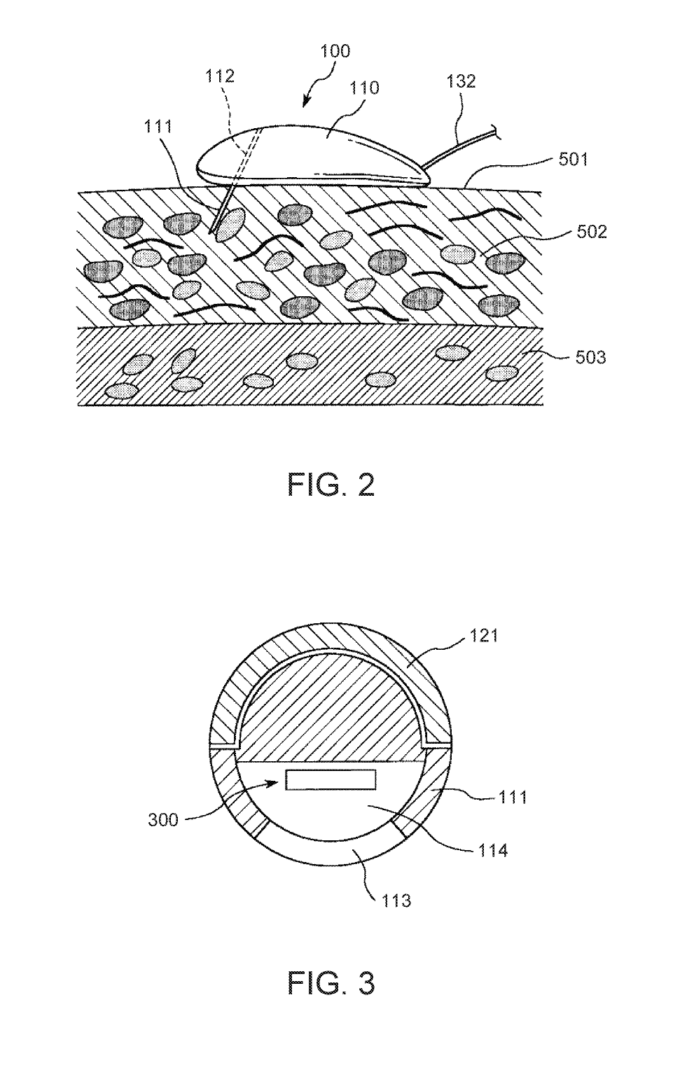

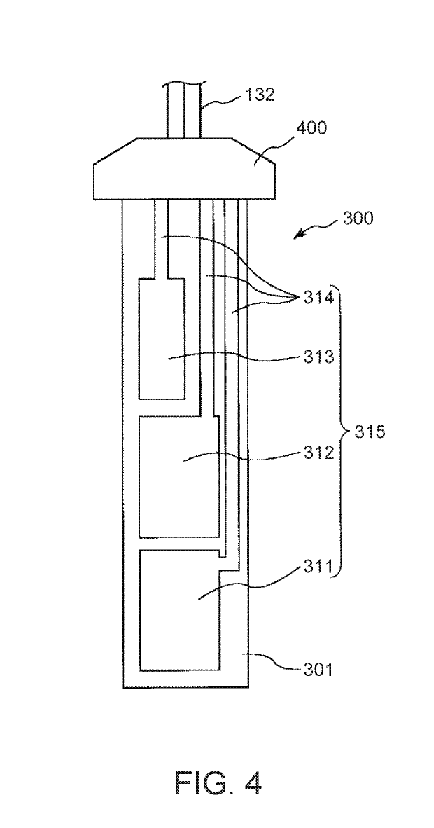

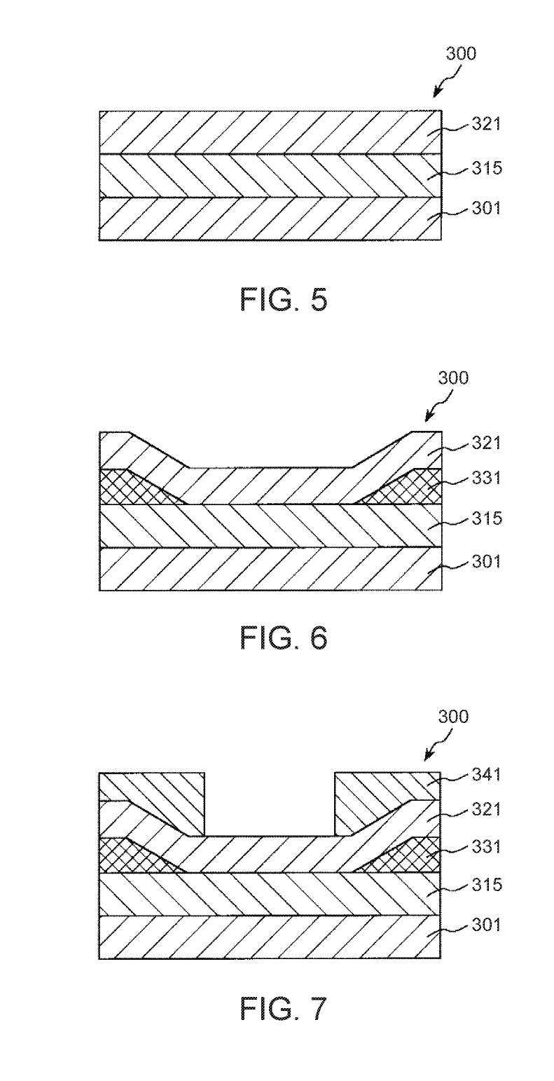

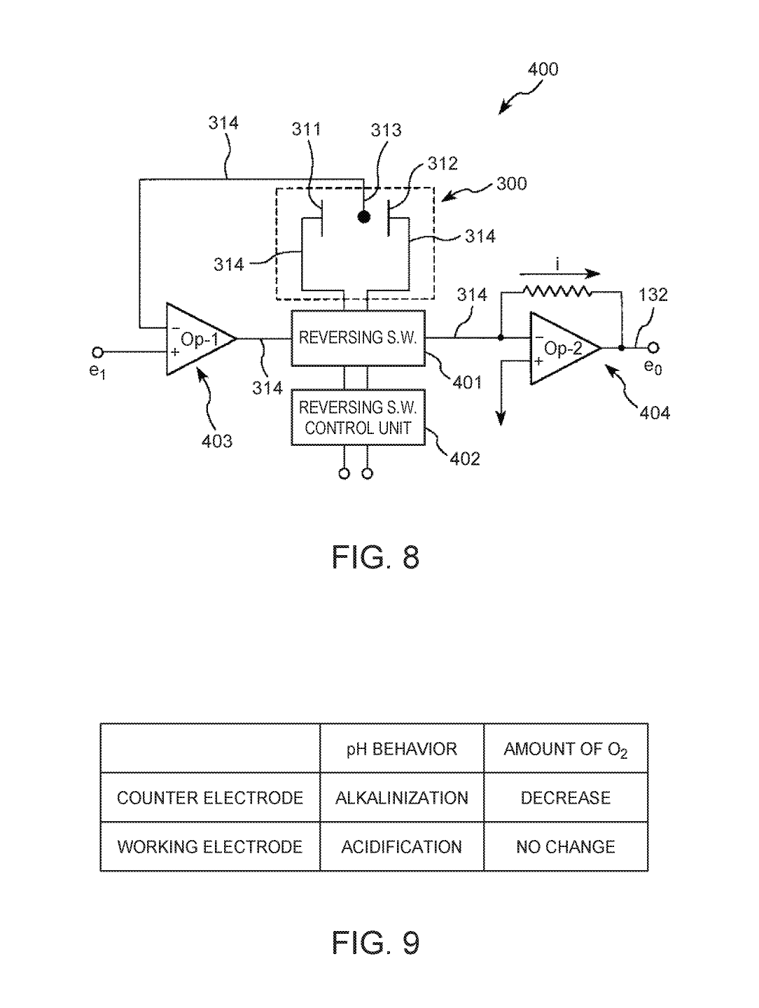

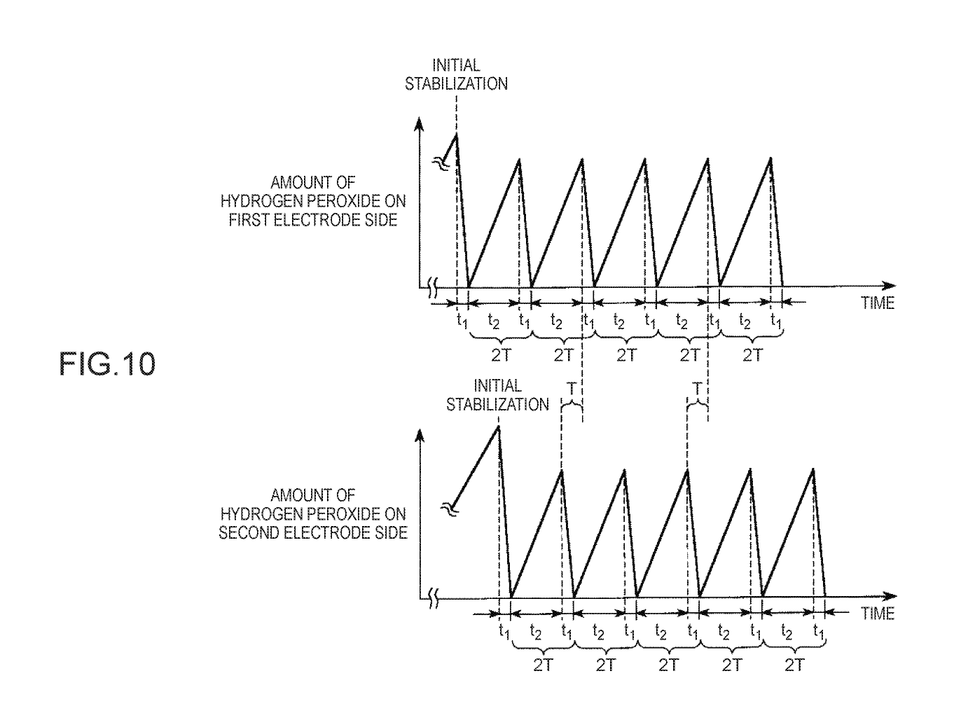

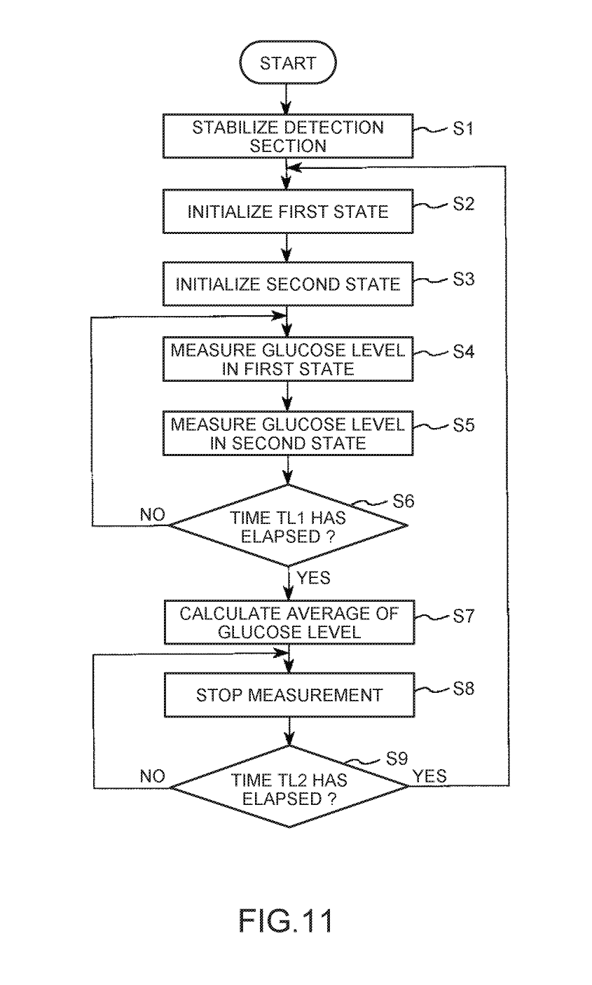

FIGS. 1A and 1B are perspective views each schematically showing a state where a detection element according to the invention is attached to a measurement device; FIG. 2 is a side view showing a state where the detection element according to the invention is attached to the skin; FIG. 3 is an enlarged vertical cross-sectional view showing a cannula included in the detection element shown in FIG. 2; FIG. 4 is a plan view showing a detection section included in the detection element shown in FIG. 2; FIG. 5 is a vertical cross-sectional view showing the detection section included in the detection element shown in FIG. 2; FIG. 6 is a vertical cross-sectional view showing another configuration example of the detection element shown in FIG. 2; FIG. 7 is a vertical cross-sectional view showing another configuration example of the detection section included in the detection element shown in FIG. 2; FIG. 8 is a view schematically showing a configuration of a circuit which controls the detection element shown in FIG. 2; FIG. 9 is a view showing the behavior of the pH value and the amount of O₂ after measuring a glucose level in a counter electrode and a working electrode; FIG. 10 is a graph showing a relationship between the amount of hydrogen peroxide and the time when a glucose level was repeatedly measured at intervals of a fixed time T by the detection section included in the detection element shown in FIG. 2; and FIG. 11 is a flow chart showing a method for repeatedly measuring a glucose level at intervals of a fixed time T by the detection section included in the detection element shown in FIG. 2. In the following description, in FIGS. 2, 3, and 5 to 7, the upper side is referred to as “upper”, and the lower side is referred to as “lower”. Further, in FIG. 4, the front side of the paper is referred to as “upper”, and the rear side of the paper is referred to as “lower”.

A measurement device 101 shown in FIGS. 1A and 1B is used by connecting a detection element 100, and includes the detection element 100, a calculation section 210 including a processing circuit 200 which calculates an electric current value obtained by the detection element 100, a display section 155 including a monitor 151 which displays a measured value obtained by calculation by the calculation section 210, a connector 131 which attaches (connects) the detection element 100 to the display section 155, and a wiring 132 which connects the processing circuit 200 to the connector 131.

As shown in FIGS. 1A to 4, the detection element 100 includes a main body section 110 including a cannula 111 to be inserted into subcutaneous tissue 502 and a detachable section 120 which is attachable to and detachable from the main body section 110 and includes a needle section 121 on a tip end side.

The detachable section 120 includes a gripping section 122 located on a proximal end side and the needle section 121 located on a tip end side, and is attached to the main body section 110 by inserting the needle section 121 into a through-hole 112 provided in the main body section 110 (see FIG. 1A), and further can be detached by pulling out the needle section 121 in a state of gripping the gripping section 122 (see FIG. 1B).

The needle section (insertion needle) 121 is sharp at the tip end and has an overall shape of a half-cylinder. The needle section 121 is configured such that when the detachable section 120 is attached to the main body section 110, the needle section 121 passes through the through-hole 112 and protrudes from the lower surface of the main body section 110, so that the needle section 121 surrounds part of the side surface of the cannula 111 as shown in FIG. 3, and thus engages with the cannula 111. Therefore, when the main body section 110 is attached to the epidermis 501, the needle section 121 pierces the epidermis 501. However, at this time, the cannula 111 surrounded by the needle section 121 also pierces the epidermis 501 along with the needle section 121 and is inserted into the subcutaneous tissue 502. Then, after this insertion of the needle section 121 accompanied with the cannula 111 into the subcutaneous tissue 502, by detaching the detachable section 120 from the main body section 110 by gripping the gripping section 122, the needle section 121 can be removed (pulled out) from the subcutaneous tissue 502 in a state where the cannula 111 is inserted (left) in the subcutaneous tissue 502. In this manner, when the main body section 110 is attached to the epidermis 501, the detachable section 120 (needle section 121) is used as a guiding member for percutaneously disposing the cannula 111 protruding from the main body section 110 in the subcutaneous tissue 502.

The main body section 110 has an overall shape of a dome and includes the cannula 111 protruding from the lower surface thereof. This main body section 110 includes an adhesive layer on the lower surface and is attached (fixed) to the epidermis 501 by bringing this lower surface into contact with the epidermis 501. At this time, the cannula 111 is disposed in the subcutaneous tissue 502 by being guided by the needle section 121 described above.

The cannula 111 has an overall shape of a cylinder and includes a hollow section 114 constituted by a communication hole (through-hole) communicating from the proximal end to the tip end and also includes a window section 113 which opens the hollow section 114 to the outside of the cannula 111.

In the hollow section 114, a detection section 300 included in the main body section 110 is disposed. The interstitial fluid contained in the subcutaneous tissue 502 by being transferred from a blood vessel 503 thereto comes in contact with this detection section 300 through the window section 113. Due to this, by the detection section 300, glucose in the interstitial fluid is detected. Then, by attaching the main body section 110 to the epidermis 501, the cannula 111 can be disposed in the subcutaneous tissue 502 over a long period of time, and therefore, the detection of glucose in the interstitial fluid can be continuously performed. That is, the main body section 110 (detection element 100) is used in CGMS (continuous glucose monitoring system) which continuously observes a glucose level in the interstitial fluid.

Here, the detection of glucose by the detection section 300 is performed using a principle as described below.

That is, in the presence of an enzyme (for example, glucose oxidase), when glucose and oxygen are present in the vicinity of the enzyme, gluconic acid and hydrogen peroxide are generated by an enzymatic reaction as shown in the following formula (1). Then, by measuring the amount of an electric current generated by electrical decomposition by applying a voltage (for example, 0.6 V) to the generated hydrogen peroxide, the amount of hydrogen peroxide can be quantitatively determined. Therefore, based on this quantitatively determined amount of hydrogen peroxide, the glucose level can be calculated.

When hydrogen peroxide is electrically decomposed, on a working electrode (anode) side, protons, oxygen, and electrons are generated by the electrical decomposition of hydrogen peroxide as shown in the following formula (2), and on a counter electrode (cathode) side, hydroxide ions are generated by reacting electrons supplied from the working electrode, and oxygen and water present in the vicinity of the electrode with one another as shown in the following formula (3).

Enzymatic reaction: glucose+O₂+H₂O→gluconic acid+H₂O₂ (1)

Working electrode: H₂O₂→O₂+2H⁺+2e⁻ (2)

Counter electrode: O₂+H₂O+4e⁻→4OH⁻ (3)

Hereinafter, the detection section 300 which detects glucose using such a principle will be described in detail.

As shown in FIGS. 4 and 5, the detection section 300 includes a substrate 301, an electrode layer 315, and a detection layer 321.

The substrate (base substrate) 301 supports the respective components (in this embodiment, the electrode layer 315 and the detection layer 321) constituting the detection section 300.

As the constituent material of the substrate 301, a variety of materials can be used without being particularly limited as long as it does not chemically react with air, water, body fluid, blood, or interstitial fluid, and is stable. Specific examples thereof include inorganic materials such as glass and SUS, and resin materials such as amorphous polyarylate (PAR), polysulfone (PSF), polyethersulfone (PES), polyphenylene sulfide (PPS), polyether ether ketone (PEEK, another name: aromatic polyether ketone), polyimide (PI), polyetherimide (PEI), fluororesins (fluorocarbon polymers), nylon, polyamides (PA) including amides, and polyesters such as polyethylene terephthalate (PET), and among these, one type or two or more types in combination can be used.

The electrode layer 315 detects electrons generated by electrically decomposing hydrogen peroxide generated in the below-mentioned detection layer 321, and measures the detected electrons as an electric current amount.

This electrode layer 315 is formed on the substrate 301 and includes a first electrode 311, a second electrode 312, a reference electrode (third electrode) 313, and a wiring 314.

The respective electrodes 311, 312, and 313 are independently electrically connected to the processing circuit 200 through the wiring 314, the wiring 132, and the connector 131. According to this, an electric current value measured by the respective electrodes 311 and 312 (electrode layer 315) is transmitted to the processing circuit 200 through the circuit 400 and the wiring 314 included in the main body section 110, and a glucose level in the interstitial fluid is calculated as a measured value by an analysis by the calculation section 210 including the processing circuit 200. Then, this measured value (glucose level) is displayed on the monitor 151, and thus, a wearer is continuously informed of the glucose level.

In the invention, the first electrode 311 and the second electrode 312 are configured to be switchable such that when the first electrode 311 functions as a working electrode, the second electrode 312 functions as a counter electrode, and when the first electrode 311 functions as a counter electrode, the second electrode 312 functions as a working electrode. The details will be described later.

The constituent materials of the respective electrodes 311, 312, and 313 are not particularly limited as long as they can be used as an enzyme electrode, and examples thereof include metal materials such as gold, silver, platinum, and alloys containing these metals, metal oxide-based materials such as ITO, and carbon-based materials such as carbon (graphite).

The deposition of the respective electrodes 311, 312, and 313 can be performed by a sputtering method, a plating method, or a vacuum heating vapor deposition method in the case where the respective electrodes 311, 312, and 313 are constituted by platinum, gold, or an alloy thereof. Further, in the case where the respective electrodes 311, 312, and 313 are constituted by carbon graphite, the deposition can be achieved by mixing carbon graphite in a binder dissolved in a suitable solvent and applying the resulting mixture.

The detection layer 321 is formed by being stacked on the electrode layer 315, that is, formed so as to cover the first electrode 311, the second electrode 312, and the reference electrode 313, and generates hydrogen peroxide from glucose permeating from the interstitial fluid coming in contact with the upper surface of the detection layer 321 by an enzymatic reaction, and supplies the hydrogen peroxide to the above-mentioned electrode layer 315.

This detection layer 321 is a layer formed to contain an enzyme, and as described above, the detection element 100 detects glucose in the interstitial fluid, and therefore, as the enzyme, glucose oxidase (GOD) is preferably used. By using glucose oxidase, the enzymatic reaction represented by the above formula (1) can be made to proceed with high activity, and therefore, hydrogen peroxide can be reliably generated from glucose in the presence of O₂ and H₂O.

Further, in the detection layer 321, a resin material is contained for the purpose of retaining the enzyme in the detection layer 321.

The resin material is not particularly limited, but, for example, methyl cellulose (MC), acetyl cellulose (cellulose acetate), polyvinylpyrrolidone (PVP), polyvinyl alcohol (PVA), polyvinyl alcohol-polyvinyl acetate copolymer (PVA-PVAc), or the like is preferably used, and among these, one type or two or more types in combination can be used. By using these resin materials, a decrease in the activity of the enzyme can be properly suppressed.

Further, in the detection layer 321, a binder or a curing agent, and other than this, albumin, a phosphate buffer, or the like may be contained.

Examples of the binder or the curing agent include a material having two or more functional groups such as aldehyde and isocyanate per molecule. By including such a binder or a curing agent in the detection layer 321, the detection layer 321 can retain the enzyme in the detection layer 321 at a high retention ratio.

Specific examples of the binder or the curing agent include glutaraldehyde, toluene diisocyanate, and isophorone diisocyanate, and among these, one type or two or more types in combination can be used. Further, examples of the binder or the curing agent utilizing UV curability include a poly(vinyl alcohol)-styrylpyridinium compound (PVA-SbQ).

The detection layer 321 containing such a binder or a curing agent can be obtained by curing a resin composition in which the binder or the curing agent, a resin material having a functional group capable of binding to a functional group contained in the binder or the curing agent, specifically, a hydroxy group, an amino group, an epoxy group, or the like at an end, and the enzyme are mixed with one another.

Further, examples of the albumin include human albumin and bovine albumin, and by including albumin, the protection and stabilization of the enzyme can be achieved.

Further, by including a phosphate buffer or the like, a variation in pH due to the enzymatic reaction can be suppressed.

The detection layer 321 may be a layer constituted by a plurality of (two or more) layers other than a layer constituted by one layer as described above.

Examples of such a detection layer 321 constituted by a plurality of (two or more) layers include a layer including at least one layer selected from a (permeation) control layer, a noise removal layer, and an (enzyme) protective layer, which is stacked on the upper or lower side of the detection layer.

The (permeation) control layer is stacked on the upper side of the (glucose) detection layer, and by this control layer, while suppressing or preventing the detection layer from coming in contact with a measurement target (interstitial fluid, and further blood), a function to allow oxygen and glucose to permeate is exhibited, and moreover, the layer has a function to control the permeability of oxygen and glucose.

Further, the (permeation) control layer having such a function is preferably capable of allowing oxygen to permeate more than glucose. According to this, in the detection of glucose using the reactions shown in the above formulae (1) to (3), an apparent decrease in the measured glucose level due to a deficiency of oxygen can be properly suppressed or prevented. That is, the improvement of the detection accuracy of the measured glucose level can be achieved.

This (permeation) control layer is not particularly limited, but examples thereof include a layer in which a cross-linked structure is constructed by forming a urethane bond using a cross-linking agent such as an isocyanate compound and also using polyethylene glycol (PEG) which is a polymer having a terminal hydroxy group, 4-hydroxybutyl acrylate, and the like alone or in admixture.

Further, other than this, a layer constituted by aminopropyl polysiloxane or the like, in which a urea resin is formed using isocyanate and an amino group, is preferably used, and a layer constituted by a siloxane resin is more preferably used, and a layer constituted by polydimethylsiloxane is particularly preferably used.

The noise removal layer is stacked on the lower side of the (glucose) detection layer and has a function to prevent a decrease in the detection sensitivity for the measured glucose level caused by permeation of a compound such as acetaminophen, ascorbic acid, or uric acid, which may be contained in the interstitial fluid through the detection layer 321 to reach the electrode layer 315.

The constituent material of the noise removal layer is not particularly limited as long as it can exhibit the above-mentioned function, but examples thereof include methyl cellulose (MC), acetyl cellulose (cellulose acetate), polyvinylpyrrolidone (PVP), polyvinyl alcohol (PVA), polyvinyl alcohol-polyvinyl acetate copolymer (PVA-PVAc), hydroxyethyl methacrylate, and poly(2-hydroxyethyl methacrylate) (HEMA), and among these, one type or two or more types in combination can be used.

In order to insolubilize the noise removal layer, an isocyanate-based compound may be added to the noise removal layer so that isocyanate can be used as a functional group, or a poly(vinyl alcohol)-styrylpyridinium compound (PVA-SbQ) or the like may be added to the noise removal layer so that UV curability can be utilized.

The noise removal layer may further contain albumin. According to this, the lower boundary surface (lower surface) of the (glucose) detection layer located on the noise removal layer can be protected.

The protective layer is a layer which is stacked on the (glucose) detection layer. In the case where the detection layer 321 includes the (permeation) control layer, this protective layer is located between the (glucose) detection layer and the (permeation) control layer.

This protective layer has a function to protect the upper boundary surface (upper surface) of the (glucose) detection layer located under the protective layer.

The constituent material of this protective layer is not particularly limited as long as it can exhibit the above-mentioned function, but examples thereof include methyl cellulose (MC), acetyl cellulose (cellulose acetate), polyvinylpyrrolidone (PVP), polyvinyl alcohol (PVA), and polyvinyl alcohol-polyvinyl acetate copolymer (PVA-PVAc), and among these, one type or two or more types in combination can be used.

The protective layer may further contain a binder or a curing agent, albumin, or the like.

Examples of the binder or the curing agent include a material having two or more functional groups such as aldehyde and isocyanate per molecule.

Specific examples of the binder or the curing agent include glutaraldehyde, toluene diisocyanate, and isophorone diisocyanate, and among these, one type or two or more types in combination can be used. Further, examples of the binder or the curing agent utilizing UV curability include a poly(vinyl alcohol)-styrylpyridinium compound (PVA-SbQ).

The protective layer containing such a binder or a curing agent can be obtained by curing a resin composition in which the binder or the curing agent, and a resin material having a functional group capable of binding to a functional group contained in the binder or the curing agent, specifically, a hydroxy group, an amino group, an epoxy group, or the like at an end are mixed with each other.

Further, examples of the albumin include human albumin and bovine albumin, and by including albumin, the protection and stabilization of the enzyme on the upper boundary surface (upper surface) of the (glucose) detection layer can be achieved.

Incidentally, a case where the detection section 300 has a three-layer structure including the substrate 301, the electrode layer 315, and the detection layer 321 as shown in FIG. 5 has been described, however, the structure is not limited thereto, and for example, the detection section 300 may have a four-layer structure further including a partition layer 331 as shown in FIG. 6, or may have a fifth-layer structure further including a partition layer 331 and an opening layer 341 as shown in FIG. 7. Hereinafter, these sections will be described in detail.

The detection section 300 having a four-layer structure shown in FIG. 6 further includes a partition layer 331 in addition to the substrate 301, the electrode layer 315, and the detection layer 321.

This partition layer 331 is stacked on the electrode layer 315, that is, inserted between the electrode layer 315 and the detection layer 321, and forms a partition including an opening section at a position corresponding to each of the first electrode 311, the second electrode 312, and the reference electrode 313 included in the electrode layer 315. By this partition layer 331, in the thickness direction of the detection section 300, regions corresponding to the respective electrodes 311, 312 and 313 are partitioned. That is, a region to which hydrogen peroxide generated by the enzymatic reaction of glucose is supplied from the detection layer 321 is strictly partitioned for the respective electrodes 311, 312 and 313. As a result, the signal noise and the effects of the reactions shown in the above formulae (2) and (3) between the respective adjacent electrodes 311, 312 and 313 can be reduced.

Further, the partition layer 331 can also be made to exhibit a function as an edge cover and/or a bank in addition to a function as a partition.

By making the partition layer 331 exhibit a function as an edge cover, the corrosion of the respective electrodes 311, 312 and 313 from an edge portion can be suppressed or prevented.

Further, by making the partition layer 331 exhibit a function as a bank, in the case where the deposition of the detection layer 321 is performed using a liquid-phase deposition method, when a liquid material for depositing the detection layer 321 is supplied to the opening section of the partition layer 331, the size of a liquid pool of this liquid material can be easily regulated.

The height, thickness, and constituent material of such a partition layer 331 are appropriately selected according to the purpose described above.

The method for forming the partition layer 331 is not particularly limited, and examples thereof include a photolithography method using a negative-type or positive-type photosensitive material containing a polyimide resin, an acrylic resin, or the like, and a photoresist method using a dry resist sheet.

The detection section 300 having a five-layer structure shown in FIG. 7 further includes a partition layer 331 and an opening layer 341 in addition to the substrate 301, the electrode layer 315, and the detection layer 321.

This partition layer 331 has the same configuration and exhibits the same function as those of the partition layer 331 described for the detection section 300 having a four-layer structure shown in FIG. 6.

Further, the opening layer 341 is stacked on the detection layer 321 and forms a partition including an opening section at a position corresponding to each of the first electrode 311, the second electrode 312, and the reference electrode 313 included in the electrode layer 315. By this opening layer 341, in the same manner as the partition layer 331, in the thickness direction of the detection section 300, regions corresponding to the respective electrodes 311, 312 and 313 are strictly partitioned. According to this, glucose permeating into the detection layer 321 from the interstitial fluid can be limited to only glucose placed in the opening section of the opening layer 341, that is, glucose supplied from the thickness direction (upward direction) of the detection layer 321, and glucose can be properly suppressed or prevented from permeating from an oblique direction. As a result, the glucose level can be more accurately detected.

The method for forming such an opening layer 341 is not particularly limited, and for example, in the case where the opening layer 341 is constituted by titanium, a method for depositing titanium using a mask sputtering method can be used. Further, in the case where the opening layer 341 is constituted by a resin material such as a naphthoquinoneazide polymer (polysulfonic acid ester), a photolithography method using a negative-type photosensitive material containing such a resin material can be used.

Further, in addition to the configurations shown in FIGS. 4 to 6, the detection section 300 can also be configured such that the electrode layer 315 includes the first electrode 311, the second electrode 312, and the reference electrode (third electrode) 313, and a wiring layer including the wiring 314 independently electrically connected to these respective electrodes 311, 312, and 313 is individually provided between the substrate 301 and the electrode layer 315.

Further, in this embodiment, a case where, in the detection section 300, all of the first electrode 311, the second electrode 312, the reference electrode 313, and the wiring 314 included in the electrode layer 315 are covered by the detection layer 321 has been described, however, the configuration is not limited thereto, and the detection layer 321 may be formed selectively on the first electrode 311 and the second electrode 312.

Further, as shown in FIG. 8, the main body section 110 has a circuit 400 which controls the detection element 100 disposed therein.

This circuit 400 includes a reversing switch 401 which switches a working electrode and a counter electrode between the first electrode 311 and the second electrode 312, a reversing switch control unit 402 which controls the operation of the reversing switch 401, an amplifier 403 for applying an amplified constant voltage between the reference electrode 313 and a working electrode, and an amplifier 404 which amplifies the value of an electric current flowing between a working electrode and a counter electrode.

The reversing switch 401 is independently electrically connected to the first electrode 311 and the second electrode 312 through the wiring 314, and is configured to be able to switch the electrical connection to the reference electrode 313 through the amplifier 403 between the first electrode 311 and the second electrode 312 by the operation of the reversing switch control unit 402.

The reversing switch control unit 402 is electrically connected to an IC (not shown) and controls the operation of the reversing switch 401 according to the program or the like previously set in this IC. According to this, by operating the reversing switch 401 by the reversing switch control unit 402, the electrical connection to the reference electrode 313 is switched between the first electrode 311 and the second electrode 312. That is, a first state where the first electrode 311 is made to function as a working electrode and the second electrode 312 is made to function as a counter electrode, and a second state where the first electrode 311 is made to function as a counter electrode and the second electrode 312 is made to function as a working electrode can be switched to each other.

The amplifier 403 is electrically connected to the reference electrode 313 and the reversing switch 401 through the wiring 314, and applies a constant voltage amplified to, for example, 0.6 V between the electrode functioning as a working electrode among the first electrode 311 and the second electrode 312 and the reference electrode 313 according to the switching by the reversing switch 401 so as to electrically decompose hydrogen peroxide to generate electrons.

The formation of the reference electrode 313 can be omitted, however, in such a case, the amplifier 403 may be electrically connected so that a constant voltage can be applied between the first electrode 311 and the second electrode 312.

Further, the amplifier 404 is electrically connected to the reversing switch 401 through the wiring 314, and amplifies the value of an electric current flowing between the first electrode 311 and the second electrode 312, that is, between a working electrode and a counter electrode, and transmits the amplified electric current value to the processing circuit 200 through the wiring 132.

Here, for example, in a detection element in the related art which does not include the reversing switch 401 or the reversing switch control unit 402, one of the first electrode 311 and the second electrode 312 becomes a working electrode, and the other becomes a counter electrode, and these are never switched to each other. In such a detection element in the related art, as described in the above background, the behavior of whether the blood glucose level increases or decreases over time with respect to the blood glucose level when the measurement is started can be determined, but the magnitude of a blood glucose level cannot be measured over time with high accuracy because of a too large drift phenomenon.

Such a drift phenomenon is presumed to occur mainly due to the following factors.

That is, in a CGMS (continuous glucose monitoring system) which continuously observes a glucose level in the interstitial fluid by disposing a detection section provided in a cannula in the subcutaneous tissue 502 over a long period of time, the cannula is inserted into the subcutaneous tissue 502 and stabilized, and thereafter, a glucose level is repeatedly measured at intervals of a fixed time T. During this fixed time T, from the interstitial fluid coming in contact with the detection layer, glucose contained in the interstitial fluid permeates into the detection layer. Then, by reacting glucose permeating during the fixed time T along with oxygen by the action of glucose oxidase contained in the detection layer, gluconic acid and hydrogen peroxide are generated as shown in the above formula (1). Then, by applying a constant voltage between a reference electrode and a working electrode, on the working electrode side, protons, oxygen, and electrons are generated by the electrical decomposition of hydrogen peroxide generated in the detection layer 321 as shown in the above formula (2), and on the counter electrode side, hydroxide ions are generated by reacting electrons supplied from the working electrode, and oxygen and water present in the vicinity of the electrode with one another as shown in the above formula (3).

Then, by measuring an electric current value between the working electrode and the counter electrode, a glucose level in the interstitial fluid can be indirectly measured based on this electric current value. However, I) as described above, in the above formulae (1) and (3), oxygen decreases on the counter electrode side (see FIG. 9). Due to this, when a glucose level is measured after the next fixed time T has elapsed while maintaining this state, a sufficient amount of oxygen may sometimes not be present in the detection layer 321 during the reaction shown in the above formula (1). As a result, unreacted glucose remains, and thus, the concentration of hydrogen peroxide generated decreases with respect to the actual glucose level, and therefore, an apparent glucose level in the interstitial fluid to be measured decreases. Further, II) as described above, in the reaction shown in the above formula (2), on the working electrode side, protons are generated, and therefore the pH in the vicinity of the working electrode decreases. Further, in the reaction shown in the above formula (3), on the counter electrode side, hydroxide ions are generated, and therefore, the pH in the vicinity of the counter electrode increases. That is, acidification occurs in the vicinity of the working electrode, and alkalinization occurs in the vicinity of the counter electrode (see FIG. 9). In general, the pH of the interstitial fluid is about 7.4, and therefore, in the initial measurement of a glucose level, the pH in the vicinity of the working electrode is also about 7.4. However, it is considered that when the measurement of a glucose level is performed a plurality of times at intervals of a fixed time T, the pH in the vicinity of the working electrode decreases and reaches 5.8 to 6.0, which is the optimal pH of glucose oxidase. As a result, the enzymatic activity of glucose oxidase increases to increase the amount of hydrogen peroxide in the detection layer, and therefore, an apparent glucose level in the interstitial fluid increases.

As described above, a drift phenomenon is considered to occur due to the main factors I) and II), however, as shown in FIG. 9, in the counter electrode and the working electrode, due to the factor II), alkalinization and acidification occur, respectively, so that the pH of the counter electrode increases, and the pH of the working electrode decreases, and therefore, the direction of pH change is in an inverse relationship.

In light of this, in the invention, as described above, a first state where the first electrode 311 is made to function as a working electrode and the second electrode 312 is made to function as a counter electrode, and a second state where the first electrode 311 is made to function as a counter electrode and the second electrode 312 is made to function as a working electrode can be switched to each other. According to this, when the first electrode 311 and the second electrode 312 are switched from a working electrode to a counter electrode, or from a counter electrode to a working electrode, respectively, these electrodes are neutralized, and therefore, the occurrence of a deviation from a pH of about 7.4, which is the pH of the interstitial fluid can be properly suppressed or prevented.

Further, as for the factor I), for example, in the case where switching between the first state and the second state is performed every time when a glucose level is measured at intervals of a fixed time T, a decrease in oxygen occurs every 2×T times when the first electrode 311 and the second electrode 312 are made to function as a counter electrode. Due to this, at intervals of this time 2×T, oxygen can be allowed to permeate into the detection layer 321 from the interstitial fluid, and as a result, the ratio of decrease in oxygen in the counter electrode can be decreased.

Accordingly, by adopting a configuration in which a first state where the first electrode 311 is made to function as a working electrode and the second electrode 312 is made to function as a counter electrode, and a second state where the first electrode 311 is made to function as a counter electrode and the second electrode 312 is made to function as a working electrode can be switched to each other as in the invention, the magnitude of a blood glucose level can be measured over time with high accuracy.

Further, in the invention, as described above, in the first state, the first electrode 311 functions as a working electrode and the second electrode 312 functions as a counter electrode, and in the second state, the first electrode 311 functions as a counter electrode and the second electrode 312 functions as a working electrode, and therefore, it is necessary to use the first electrode 311 and the second electrode 312 as electrodes having the same function in the different states. Due to this, the first electrode 311 and the second electrode 312 preferably have the same configuration.

Specifically, the first electrode 311 and the second electrode 312 are preferably configured such that the upper surfaces thereof are covered by the detection layer in the electrode layer 315 as in this embodiment.

Further, the areas of the upper surfaces of the first electrode 311 and the second electrode 312 preferably have the same size.

By satisfying these conditions, it can be said that the first electrode 311 and the second electrode 312 have the same configuration, and both of the first electrode 311 and the second electrode 312 reliably exhibit both functions of a working electrode and a counter electrode.

Further, the measurement of a glucose level in the interstitial fluid using the detection section 300 configured to be able to switch the first state where the first electrode 311 is made to function as a working electrode and the second electrode 312 is made to function as a counter electrode, and a second state where the first electrode 311 is made to function as a counter electrode and the second electrode 312 is made to function as a working electrode to each other as described above is specifically performed, for example, as follows.

Incidentally, hereinafter, a case where a glucose level (electric current value) is repeatedly measured at intervals of a fixed time T, and switching between the first state and the second state is performed every time when this measurement is performed will be described as an example.

[1] First, the cannula 111 is inserted into the subcutaneous tissue 502 and the detection layer 321 is brought into contact with the interstitial fluid and stabilized (S1).

[2] Subsequently, by operating the reversing switch 401 by the reversing switch control unit 402, the state is put into the first state where the first electrode 311 is made to function as a working electrode and the second electrode 312 is made to function as a counter electrode. Then, in this first state, a constant voltage is applied between the first electrode 311 functioning as a working electrode and the reference electrode 313 for a time length of t₁. According to this, hydrogen peroxide generated in the vicinity of the first electrode 311 of the detection layer 321 during stabilization is electrically decomposed as shown in the above formula (2), and as a result, the vicinity of the first electrode 311 of the detection layer 321 is initialized (S2).

[3] Subsequently, by operating the reversing switch 401 by the reversing switch control unit 402, the state is put into the second state where the first electrode 311 is made to function as a counter electrode and the second electrode 312 is made to function as a working electrode. Then, in this second state, a constant voltage is applied between the second electrode 312 functioning as a working electrode and the reference electrode 313 for a time length of t₁. According to this, hydrogen peroxide generated in the vicinity of the second electrode 312 of the detection layer 321 during stabilization is electrically decomposed as shown in the above formula (2), and as a result, the vicinity of the second electrode 312 of the detection layer 321 is initialized (S3).

A difference between the start time when the application of the constant voltage between the first electrode 311 and the reference electrode 313 is started in the above step [2] and the start time when the application of the constant voltage between the second electrode 312 and the reference electrode 313 is started in this step [3] is set to a fixed time T (see FIG. 10).

[4] Subsequently, by operating the reversing switch 401 by the reversing switch control unit 402, the state is put into the first state where the first electrode 311 is made to function as a working electrode and the second electrode 312 is made to function as a counter electrode. Then, in the first state, a constant voltage is applied between the first electrode 311 functioning as a working electrode and the reference electrode 313 for a time length of t₁ at intervals such that a difference between the start time when the application of the constant voltage between the first electrode 311 and the reference electrode 313 is started in the above step [2] and the start time when the application of the constant voltage between the first electrode 311 and the reference electrode 313 is started in this step [4] is set to a fixed time 2T, that is, when a time during which the constant voltage is not applied between the first electrode 311 and the reference electrode 313 is set to t₂, the following relationship is satisfied: t₂=2T−t₁ (see FIG. 10). According to this, hydrogen peroxide generated in the vicinity of the first electrode 311 of the detection layer 321 during the time t₂ is electrically decomposed as shown in the above formula (2), and by measuring the thus generated electrons as the value of an electric current flowing between the first electrode 311 and the second electrode 312, a glucose level in the interstitial fluid can be obtained (S4).

[5] Subsequently, by operating the reversing switch 401 by the reversing switch control unit 402, the state is put into the second state where the first electrode 311 is made to function as a counter electrode and the second electrode 312 is made to function as a working electrode. Then, in the second state, a constant voltage is applied between the second electrode 312 functioning as a working electrode and the reference electrode 313 for a time length of t₁ at intervals such that a difference between the start time when the application of the constant voltage between the second electrode 312 and the reference electrode 313 is started in the above step [3] and the start time when the application of the constant voltage between the second electrode 312 and the reference electrode 313 is started in this step [5] is set to a fixed time 2T, that is, when a time during which the constant voltage is not applied between the second electrode 312 and the reference electrode 313 is set to t₂, the following relationship is satisfied: t₂=2T−t₁ (see FIG. 10). According to this, hydrogen peroxide generated in the vicinity of the second electrode 312 of the detection layer 321 during the time t₂ is electrically decomposed as shown in the above formula (2), and by measuring the thus generated electrons as the value of an electric current flowing between the first electrode 311 and the second electrode 312, a glucose level in the interstitial fluid can be obtained (S5).

[6] Subsequently, the above steps [4] and [5] are repeated until a predetermined time TL1 is reached (S6), and an average of the glucose level measured during this time is obtained (S7), whereby the glucose level is averaged, and therefore can be obtained with high reliability.

Incidentally, after the time TL1 has elapsed, the measurement of a glucose level in the above steps [4] and [5] is stopped (S8).

Even when the measurement of a glucose level is performed a plurality of times in this manner, by performing switching between the first state and the second state, as described above, the occurrence of the factors I) and II) can be properly suppressed or prevented, and based on hydrogen peroxide generated on each electrode side, a glucose level can be measured with high accuracy (see FIG. 10). That is, by suppressing the occurrence of a drift phenomenon, the magnitude of a blood glucose level in the interstitial fluid can be measured over time with high accuracy.

[7] Further, the above steps [2] to [6] are performed every time when a predetermined time TL2 has elapsed (S9). According to this, a glucose level (an average of the glucose level at intervals of a time TL2) is obtained at intervals of a time TL2, and therefore, a glucose level can be measured continuously with high reliability.

In this embodiment, a case where, when a glucose level is repeatedly measured at intervals of a fixed time T, switching between the first state and the second state is performed every time when this measurement is performed has been described, however, the embodiment is not limited thereto, and for example, after a glucose level is measured a plurality of times (two or more times), switching between the first state and the second state is performed, and two or more times of measurement of a glucose level may be determined to be a fixed number of times. Also in such a detection method, the same effect as in the case where switching between the first state and the second state is performed every time when a glucose level is measured can be obtained.

That is, in the repeated measurement of a glucose level at intervals of a fixed time T as described above, switching between the first state and the second state may be performed after performing the measurement of a glucose level a fixed number of times.

By performing the steps as described above, a glucose level (glucose concentration) in the interstitial fluid is measured.

### Insulin Supply Device

The detection element of the invention is attached to a measurement device as described above, and in addition thereto, also attached to, for example, an insulin supply device.

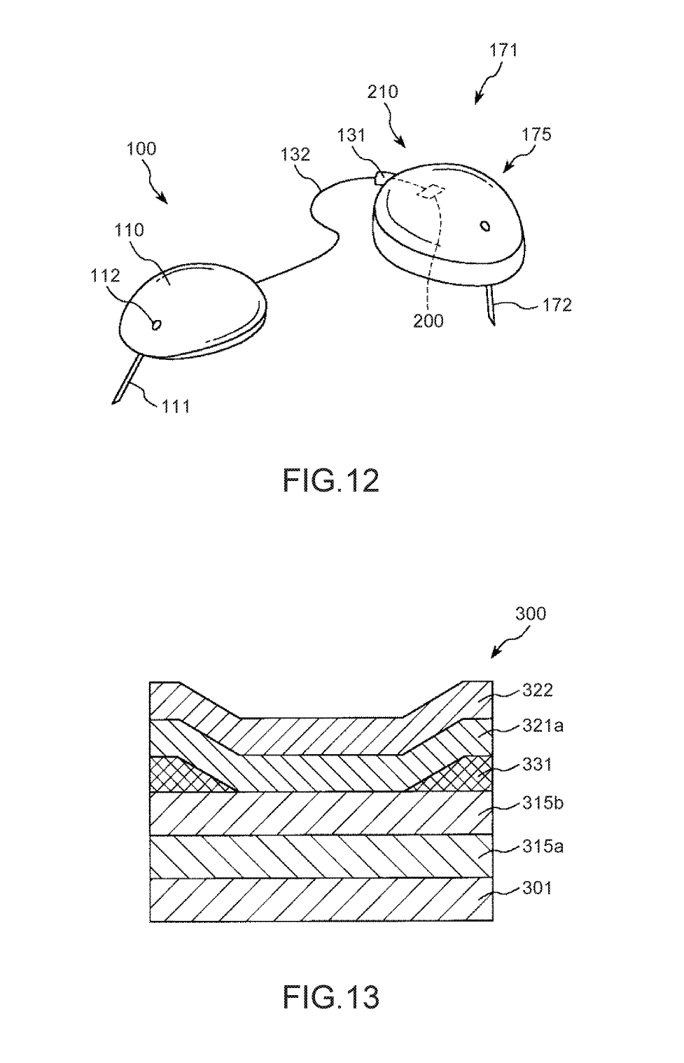

FIG. 12 is a perspective view schematically showing a state where the detection element according to the invention is attached to an insulin supply device.

An insulin supply device 171 shown in FIG. 12 is used by connecting a detection element 100, and includes the detection element 100, a calculation section 210 including a processing circuit 200 which analyzes an electric current value obtained by the detection element 100, a supply section 175 including a needle section 172 for supplying (administering) insulin to the subcutaneous tissue 502 based on the measured value obtained by calculation by the calculation section 210, a connector 131 which attaches (connects) the detection element 100 to the supply section 175, and a wiring 132 which connects the processing circuit 200 to the connector 131. In the insulin supply device 171, the value of an electric current flowing between a first electrode 311 and a second electrode 312 is transmitted to the processing circuit 200 through a circuit 400 included in a main body section 110 and the wiring 132, and by an analysis of the calculation section 210 including the processing circuit 200, a glucose level (glucose concentration) in the interstitial fluid is calculated as a measured value. Then, based on this measured value (glucose level), that is, in the case where the measured value is higher than a predetermined concentration, the insulin supply device 171 is operated, and insulin is automatically administered to a wearer through the needle section 172.

Hereinabove, the detection method using a detection element, the detection element, the measurement device, and the insulin supply device according to the invention have been described based on the embodiments shown in the drawings, however, the invention is not limited thereto.

For example, the configurations of the respective components in the detection element, the measurement device, and the insulin supply device according to the invention can be replaced with an arbitrary configuration having a similar function. In addition, another arbitrary configuration may be added to the invention. Further, the invention may be configured such that arbitrary two or more configurations (features) of the measurement device and the insulin supply device are combined.

Further, the cannula may be inserted into the skin other than inserting the cannula into the subcutaneous tissue.

Further, in the measurement device and the insulin supply device, an electric current value measured by the detection element may be transmitted to the processing circuit without passing through a wiring, and for example, an electric current value may be transmitted wirelessly to the processing circuit through a communication unit.

Further, in the measurement device, the detection element and the display section are not limited to those connected to each other through the wiring, and these members may be integrally formed, and in the insulin supply device, the detection element and the supply section are not limited to those connected to each other through the wiring, and these members may be integrally formed.

Further, to the detection method using a detection element according to the invention, one or more arbitrary steps may be added.

### EXAMPLES

Next, specific examples of the invention will be described.

### 1. Production of Detection Element

<1> First, a transparent glass substrate having an average thickness of 0.5 mm was prepared as a substrate 301. Subsequently, on this substrate 301, a patterned conductive ITO film having an average thickness of 100 nm was formed as a wiring layer 315a including a wiring 314 by a mask sputtering method.

<2> Subsequently, on the ITO film, a patterned platinum film having an average thickness of 200 nm was formed as an electrode layer 315b including a first electrode 311, a second electrode 312, and a reference electrode 313 by a mask sputtering method.

<3> Subsequently, on the platinum film, a bank having an average height of 8 μm was formed by a photoresist using an acrylic material, and also an opening section which comes in contact with a measurement target fluid (blood or interstitial fluid) was opened, whereby a partition layer 331 was formed.

<4> Subsequently, the substrate on which the conductive ITO film, the platinum film, and the partition layer were stacked was immersed in acetone and 2-propanol in this order, followed by ultrasonic cleaning, and then, an oxygen plasma treatment and an argon plasma treatment were performed. These plasma treatments were performed under the conditions that the plasma power was 100 W, the gas flow rate was 20 sccm, and the treatment time was 5 sec while heating the substrate to 70 to 90° C.

<5> Subsequently, on the platinum electrode serving as the reference electrode, an ink containing silver-silver chloride particles was applied by an inkjet method in an amount such that the film thickness after drying was 3 μm, and dried in an oven at 120° C., whereby a silver-silver chloride film was formed.

<6> Subsequently, phosphate-buffered saline (pH 7.4) was used as a solvent, and GOD (glucose oxidase, activity: 150 U/mg) at 20 KU/mL, BSA (bovine serum albumin) at 10 wt %, MC (methyl cellulose) at 10 wt %, and PVA-SbQ (a 10% aqueous solution) at 20 wt % were added thereto, whereby a detection layer forming material was prepared.

Then, this detection layer forming material was applied onto the entire surfaces of the first electrode, the second electrode, and the reference electrode by a spin coating method, and then dried, whereby a thin film having a film thickness of 2 μm was obtained. Thereafter, the film was irradiated with an ultraviolet light with a wavelength of 365 nm at 1000 mJ to form an insolubilized film, whereby a (glucose) detection layer 321a was formed.

<7> Subsequently, a mixture containing PEG-600 (polyethylene glycol) and PEG-400 (polyethylene glycol) at 1:1 at 50 wt %, aminopropyl polysiloxane at 30 wt %, 4-hydroxybutyl acrylate at 15 wt %, and isophorone diisocyanate at 5 wt % were mixed with one another, whereby a control layer forming material was prepared.

Then, this control layer forming material was applied onto the entire surface of the (glucose) detection layer by a spin coating method, and then dried, whereby a thin film having a film thickness of 2 μm was obtained. Thereafter, the film was stored at 50° C. for 10 hours to complete insolubilization, whereby a control layer 322 was formed.

By the above steps, the detection section 300 having a configuration shown in FIG. 13 was produced.

### 2. Evaluation “No Problem”

### Example 1

First, a solution prepared in such a manner that a glucose concentration in phosphate-buffered saline was 100 mg/dL was prepared as a standard solution.

Then, this standard solution was heated to a constant temperature of 35° C.

Subsequently, the detection section 300 having a configuration shown in FIG. 13 was immersed in the heated standard solution, so that the control layer 322 and the (glucose) detection layer 321a at positions corresponding to the first electrode, the second electrode, and the reference electrode were impregnated with the standard solution.

Subsequently, a voltage was applied so that the voltage between the reference electrode and the first electrode was 0.6 V, and thereafter, this state was maintained for 2 hours to stabilize the state.

Subsequently, an electric current value was measured every 10 seconds. At this time, the polarity was reversed between the first electrode and the second electrode every time when an electric current value was measured once. That is, in the odd-numbered measurement of an electric current value, the first electrode was made to function as a working electrode and the second electrode was made to function as a counter electrode, and in the even-numbered measurement of an electric current value, the first electrode was made to function as a counter electrode and the second electrode was made to function as a working electrode.

The electric current values measured in this manner were averaged every 1 hour, which was used as data, and the measurement was performed up to 48 hours.

### Comparative Example 1

The same procedure as in Example 1 was performed except that reversing of the polarity between the first electrode and the second electrode was omitted when an electric current value was measured every 10 seconds by the detection section 300 having a configuration shown in FIG. 13, that is, the measurement of an electric current value for 48 hours was performed by making the first electrode to function as a working electrode and the second electrode to function as a counter electrode.

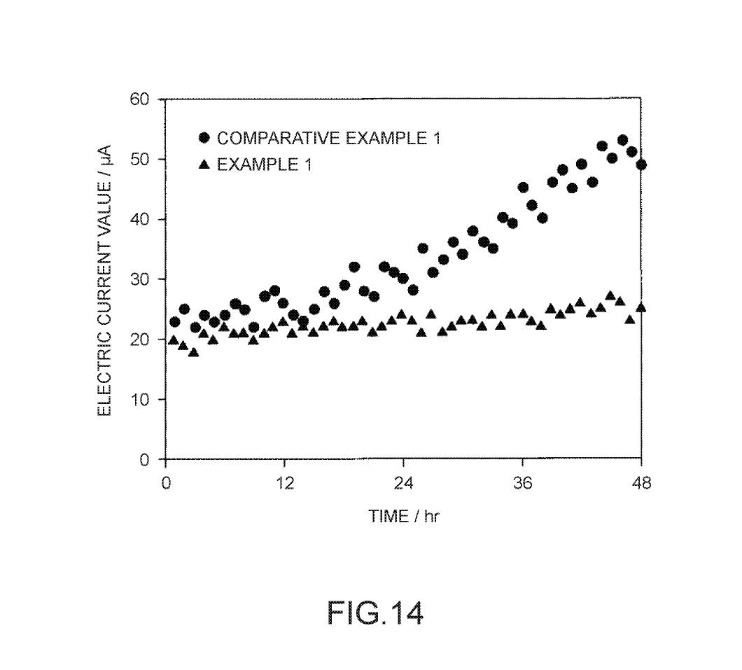

The results are shown in FIG. 14.

As apparent from FIG. 14, by reversing the polarity every time when an electric current value was measured (every 10 seconds) as in Example 1, even after 48 hours, almost no change was observed in the measured electric current value from the initially measured value.

On the other hand, in Comparative Example 1, since the polarity was not reversed every time when an electric current value was measured, the signal intensity (electric current value) increased over time.

This is considered to be because in Comparative Example 1, as the enzymatic reaction in the (glucose) detection layer 321a proceeds, the pH changes due to the reactions shown in the following formulae (2) and (3) at positions corresponding to the first electrode (working electrode) and the second electrode (counter electrode) of the detection layer 321a, and therefore, the activity of the enzyme changes.

First electrode: H₂O₂→O₂+2H⁺+2e⁻ (2)

Second electrode: O₂+H₂O+4e⁻→4OH⁻ (3)

In Comparative Example 1, it is presumed that as the time elapses, acidification proceeds on the first electrode (working electrode), and the pH changes from about 7.4 which is the initial value to an acidic pH.

Here, the optimal pH of the enzymatic activity of glucose oxidase (GOD) is from 5.8 to 6.0, and therefore, in consideration of this, it is reasonable to consider that the reason why the electric current value of data obtained in Comparative Example 1 increases over time is because the enzymatic activity increases due to the change in the pH.

On the other hand, in consideration that the pH changes to an alkaline pH over time in the second electrode (counter electrode), by reversing the polarity such that a working electrode and a counter electrode are switched between the first electrode and the second electrode as in Example 1, a neutralization reaction occurs between H⁺ and OH⁻, and the change in the pH is suppressed, and as a result, the change in the electric current value of data obtained in Example 1 is considered to be decreased.

The entire disclosure of Japanese Patent Application No. 2015-031435 filed Feb. 20, 2015 is expressly incorporated by reference herein.

What is claimed is:

1. A detection method using a detection element including a first electrode and a second electrode, in which
a detection layer containing an enzyme is provided in the first electrode and the second electrode,
the method comprises:
a first step of measuring an electric current generated by decomposition of hydrogen peroxide generated from glucose in the detection layer of the first electrode as an electric current value between the first electrode and the second electrode; and
a second step of measuring an electric current generated by decomposition of hydrogen peroxide generated from glucose in the detection layer of the second electrode as an electric current value between the first electrode and the second electrode.

2. The detection method using a detection element according to claim 1, wherein the glucose is contained in a material permeating into the detection layer from the interstitial fluid in the subcutaneous tissue and the skin.

3. The detection method using a detection element according to claim 2, wherein the level of glucose contained in the interstitial fluid is detected based on the electric current value.

4. The detection method using a detection element according to claim 1, wherein the enzyme is glucose oxidase which decomposes the glucose, oxygen, and water to gluconic acid and the hydrogen peroxide.

5. The detection method using a detection element according to claim 1, wherein the detection layer is provided in each of the first electrode and the second electrode.

6. The detection method using a detection element according to claim 5, wherein the areas of the detection layers of the first electrode and the second electrode have substantially the same size.

7. The detection method using a detection element according to claim 1, wherein a third electrode which decomposes hydrogen peroxide by applying a substantially constant voltage between the third electrode and the first electrode is included.

8. The detection method using a detection element according to claim 1, wherein switching between the first step and the second step is performed by a reversing switch which electrically reverses the first electrode and the second electrode.

9. The detection method using a detection element according to claim 1, wherein the electric current value is measured a plurality of times at intervals of a fixed time, and switching between the first step and the second step is performed every time when the electric current value is measured.

10. The detection method using a detection element according to claim 9, wherein the electric current value is measured a plurality of times at intervals of a fixed time, and the plurality of times is determined to be a fixed number of times.

11. A detection element, comprising:
a substrate;
a first electrode and a second electrode provided on the substrate;
a detection layer containing an enzyme provided in the first electrode and the second electrode; and
a reversing switch which electrically reverses the first electrode and the second electrode, wherein
an electric current generated by decomposition of hydrogen peroxide generated from glucose in the detection layer is measured as an electric current value between the first electrode and the second electrode.

12. A measurement device, comprising the detection element according to claim 11, a calculation section which calculates a glucose level from an electric current value measured by the detection element, and a display section which displays the glucose level.

13. An insulin supply device, comprising the detection element according to claim 11, a calculation section which calculates a glucose level from an electric current value measured by the detection element, and a supply section which supplies insulin to the subcutaneous tissue and into the skin based on the glucose level.
